(19) United States
(12) Patent Application Publication
(10) Pub. No.: US 2005/0215600 A1
(43) Pub. Date: Sep. 29, 2005

(54) Polymorphs of torsemide hydrochloride and process for production thereof

(76) Inventors: Arie Gutman, Haifa (IL); Marina Etinger, Nesher (IL); Dmitry Goldring, Nazareth Illite (IL); Boris Pertzikov, Haifa (IL); Gennady Nisnevich, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/974,026

(22) Filed: Oct. 27, 2004

Related U.S. Application Data
(60) Provisional application No. 60/513,989, filed on Oct. 27, 2003.

(51) Int. Cl. C07D 213/70; A61K 31/44

(52) U.S. Cl. 514/346; 546/293

(57) ABSTRACT

This invention provides a hydrochloride salt of torsemide. This invention further provides polymorphic forms of torsemide hydrochloride. Processes for preparing polymorphic forms of torsemide hydrochloride are also provided. This invention further provides a method for purifying crude torsemide or a salt thereof. Pharmaceutical composition comprising polymorphic forms of torsemide hydrochloride are also provided.

### CROSS-REFERENCE TO RELATED APPLICATIONS

This application claims priority from a Provisional Application U.S. Ser. No. 60/513,989, filed Oct. 27, 2003, which is hereby incorporated by reference.

### FIELD OF THE INVENTION

This invention provides a hydrochloride salt of torsemide. This invention further provides polymorphic forms of torsemide hydrochloride. Processes for preparing polymorphic forms of torsemide hydrochloride are also provided. This invention further provides a method for purifying crude torsemide or a salt thereof. Pharmaceutical composition comprising polymorphic forms of torsemide hydrochloride are also provided.

### BACKGROUND OF THE INVENTION

Torsemide (also known as torasemide) (N-[(isopropylamino)-carbonyl]-4-(m-tolylamino)-3-pyridinesulfonamide) [1] 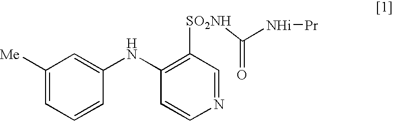
is a powerful diuretic which may be used for the treatment of hypertension and heart failure (J. Delarge, Arzneim.-Forsch./Drug Res., 1988, v. 38, 144 and U.S. Pat. No. 4,018,929) Torsemide is approved under the trade mark DEMADEX® by the U.S. Food and Drug Administration, for the treatment of hypertension and edema associated with congestive heart failure, renal disease, or hepatic disease.

### SUMMARY OF THE INVENTION

The present invention provides, in one embodiment, a hydrochloride salt of torsemide. In one embodiment, the present invention provides a solid form of torsemide hydrochloride. In another embodiment, the present invention further provides an amorphous torsemide hydrochloride. Furthermore, in another embodiment the present invention provides a crystalline form of torsemide hydrochloride. Furthermore, in another embodiment the present invention provides a solvent adduct of torsemide hydrochloride. In one embodiment, the present invention further provides a hydrate of torsemide hydrochloride.

In addition, in one embodiment, the present invention provides a process for the preparation of torsemide hydrochloride in a solid form comprising the step of contacting torsemide or a salt thereof with hydrogen chloride.

In addition, in one embodiment, the present invention provides a process for the preparation of the crystalline form of torsemide hydrochloride comprising the step of adding at least one solvent to a torsemide hydrochloride solution in an amount sufficient to induce the formation of crystalline torsemide hydrochloride precipitate, thereby obtaining the crystalline form of torsemide hydrochloride.

In addition, in one embodiment, the present invention provides a process for the preparation of the crystalline form II of torsemide hydrochloride comprising the step of adding a solvent mixture which comprises DMSO to a torsemide hydrochloride solution in an amount sufficient to induce the formation of crystalline torsemide hydrochloride precipitate, thereby obtaining the crystalline form II of torsemide hydrochloride.

In addition, in one embodiment, the present invention provides a process for the preparation of the crystalline form I of torsemide hydrochloride comprising the step of adding water to a torsemide hydrochloride solution in an amount sufficient to induce the formation of crystalline torsemide hydrochloride Form I precipitate, thereby obtaining the crystalline form I of torsemide hydrochloride.

In addition, in one embodiment, the present invention provides a process for the preparation of the crystalline form III of torsemide hydrochloride comprising the step of adding at least one water miscible organic solvent to a torsemide hydrochloride solution in an amount sufficient to induce the formation of crystalline torsemide hydrochloride Form III precipitate, thereby obtaining the crystalline form III of torsemide hydrochloride.

In addition, in one embodiment, the present invention provides a method for purifying crude torsemide or a salt thereof from related impurities comprising the steps of contacting crude torsemide or a salt thereof with hydrogen chloride to obtain torsemide hydrochloride and (re)crystallizing, trituring and/or reslurring the torsemide hydrochloride to obtain a substantially pure crystalline torsemide hydrochloride.

Furthermore, in one embodiment, the present invention provides a pharmaceutical composition comprising the hydrochloride salt of torsemide and a pharmaceutically acceptable carrier.

### BRIEF DESCRIPTION OF THE DRAWINGS

FIG. 1 shows a schematic perspective view of torsemide hydrochloride molecule and the atomic numbering of non-hydrogen atoms as derived from single crystal x-ray analysis. (Atomic coordinates based on Table 2).

FIG. 2 shows a characteristic x-ray powder diffraction pattern of torsemide hydrochloride Form II of the present invention. Vertical axis: intensity (CPS); Horizontal axis: 2 theta (degrees).

FIG. 3 shows calculated x-ray powder diffraction pattern of crystalline torsemide hydrochloride Form II. Vertical axis: intensity (CPS); Horizontal axis: 2 theta (degrees).

FIG. 4 shows the infrared (IR) spectrum of torsemide hydrochloride Form II of the present invention in potassium bromide.

FIG. 5 shows the differential scanning calorimetry (DSC) thermogram of torsemide hydrochloride Form II.

FIG. 6 shows a characteristic x-ray powder diffraction pattern of torsemide hydrochloride Form I of the present invention. Vertical axis: intensity (CPS); Horizontal axis: 2 theta (degrees).

FIG. 7 shows the infrared (IR) spectrum of torsemide hydrochloride Form I of the present invention in potassium bromide.

FIG. 8 shows a characteristic TGA of torsemide hydrochloride Form I of the present invention.

FIG. 9 shows a characteristic x-ray powder diffraction pattern of torsemide hydrochloride Form III of the present invention. Vertical axis: intensity (CPS); Horizontal axis: 2 theta (degrees).

FIG. 10 shows the infrared (IR) spectrum of torsemide hydrochloride Form III of the present invention in potassium bromide.

FIG. 11 shows a characteristic TGA of torsemide hydrochloride Form III of the present invention.

### DETAILED DESCRIPTION OF THE INVENTION

The present invention provides, in one embodiment, a hydrochloride salt of torsemide. In one embodiment, the present invention provides a solid form of torsemide hydrochloride. In one embodiment, the present invention further provides an amorphous torsemide hydrochloride. In one embodiment, an amorphous form is a non-crystalline solid form. Furthermore, in one embodiment, the present invention provides a crystalline form of torsemide hydrochloride. In another embodiment, the present invention provides polymorph forms of the crystalline torsemide hydrochloride. Furthermore, in one embodiment, the present invention provides a solvent adduct of torsemide hydrochloride. The present invention further provides, in one embodiment, a hydrate of torsemide hydrochloride.

In one embodiment, the crystalline state of a compound is described by crystallographic parameters. In one embodiment of the invention the cell parameters are unit cell dimensions, space group and atomic position of all atoms in the compound relative to the origin of its unit cell. In one embodiment of the invention, the crystallographic parameters are experimentally determined by single crystal x-ray analysis. In one embodiment of the invention, the crystal structure of torsemide hydrochloride is determined at 293 K. In one embodiment of the invention, the unit cell parameters are according to Table 1.

TABLE 1
Crystal parameters of torsemide hydrochloride Form II

| Formula | C₁₆H₂₀ClN₄O₃S |
|---|---|
| Formula weight (amu) | 383.87 |
| Space group | P-1 |
| Cell dimensions | |
| a (Å) | 11.691 |
| b (Å) | 16.649 |
| c (Å) | 18.130 |
| α (°) | 62.79 |
| β (°) | 71.25 |
| γ (°) | 79.44 |
| V (Å³) | 2969.1 |
| Z (molecules/units cell) | 6 |
| Density (g/cm³) | 1.288 |

In one embodiment of the invention, the unit cell dimension is defined by three parameters: length of the sides of the cell, relative angles of sides to each other and the volume of the cell. In another embodiment, the lengths of the sides of the unit cell are defined by a, b and c. In another embodiment, the relative angles of the cell sides are defined by α, β and γ. In another embodiment, the volume of the cell is defined as V. In one embodiment of the invention, a is 11.691 Å. In another embodiment, b is 16.649 Å. In another embodiment, c is 18.130 Å. In one embodiment of the invention, α is 62.79°. In another embodiment, β is 71.25°. In another embodiment, γ is 79.44°. In another embodiment, V is 2969.1 Å³. In one embodiment of the invention, Z is the number of molecules in a unit cell. In another embodiment, Z is 6. In one embodiment of the invention, the density of the cell is 1.288 g/cm³.

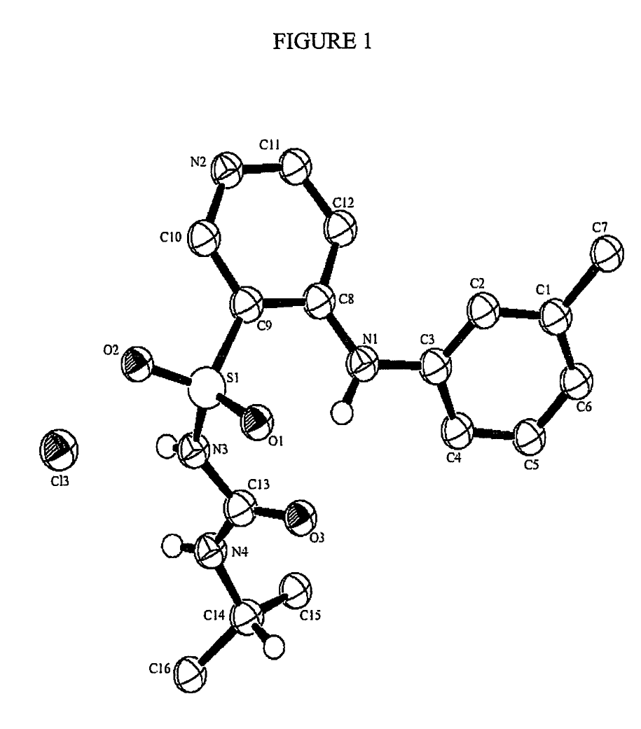

In one embodiment of the invention, the crystalline torsemide hydrochloride has a single crystal x-ray crystallographic analysis. In another embodiment, the x-ray crystallographic analysis yields atomic positions of all atoms relative to the origin of the unit cell as showed in Tables 2 through 6. In another embodiment, the atomic coordinates, and their equivalent isotropic displacement parameters are according to Tables 2. In another embodiment, the bond lengths are according to Tables 3. In another embodiment, the bond angles are according to Tables 4. In another embodiment, the anisotropic thermal parameters are according to Tables 5. In another embodiment, the proton atom coordinates and their isotropic thermal parameters are according to Tables 6. In another embodiment, the x-ray crystallographic analysis yields atomic positions of all atoms relative to the origin of the unit cell as represented in FIG. 1.

TABLE 2
Atomic coordinates (×10⁴) and equivalent isotropic displacement
parameters (Å² × 10³). U (eq) is defined as one third of the
trace of the orthogonalized Uij tensor.

| | x | y | z | U (eq) |
|---|---|---|---|---|
| C1(1) | 5003(1) | 2820(1) | 5567(1) | 62(1) |
| C1(2) | 778(1) | 3023(1) | 3586(1) | 80(1) |
| C1(3) | 2990(1) | 4931(1) | 3132(1) | 65(1) |
| C (1) | 3012(4) | 9913(3) | 1941(2) | 58(1) |
| C (2) | 2268(4) | 9347(2) | 2450(2) | 53(1) |
| C (3) | 1271(3) | 8902(2) | 2125(2) | 47(1) |
| C (4) | −972(4) | 9034(3) | 1277(2) | 61(1) |
| C (5) | −1708(4) | 9608(3) | 767(2) | 72(1) |
| C (6) | −2700(4) | 10032(3) | 1096(3) | 69(1) |
| C (7) | −4120(4) | 10372(3) | 2319(3) | 96(2) |
| C (8) | −766(3) | 7680(2) | 3427(2) | 41(1) |
| C (9) | 127(3) | 7182(2) | 3847(2) | 40(1) |
| C (10) | −238(4) | 6510(2) | 4692(2) | 49(1) |
| C (11) | −2236(3) | 6742(2) | 4669(2) | 47(1) |
| C (12) | −1962(3) | 7417(2) | 3867(2) | 43(1) |
| C (13) | 2229(3) | 7413(3) | 1859(2) | 49(1) |
| C (14) | 2955(5) | 7206(3) | 526(3) | 88(1) |
| C (15) | 1870(6) | 7156(5) | 304(3) | 160(3) |
| C (16) | 4041(6) | 6683(5) | 217(3) | 166(3) |
| N(1) | −461(3) | 8349(2) | 2627(2) | 46(1) |
| N(2) | −1392(3) | 6295(2) | 5090(2) | 51(1) |
| N(3) | 2170(2) | 6962(2) | 2737(2) | 52(1) |
| N(4) | 2739(3) | 6889(2) | 1450(2) | 66(1) |
| O(1) | 1827(2) | 8317(2) | 3031(2) | 65(1) |
| O(2) | 2226(2) | 6792(2) | 4134(2) | 71(1) |
| O(3) | 1866(2) | 8192(2) | 1518(2) | 61(1) |
| S(1) | 1674(1) | 7361(1) | 3452(1) | 53(1) |
| S(2) | 9310(1) | 5677(1) | 3011(1) | 51(1) |
| S(3) | 2664(1) | 1074(1) | 4966(1) | 50(1) |
| C(201) | 5964(5) | 8374(4) | 304(3) | 99(2) |
| C(202) | 6731(4) | 7734(3) | 775(3) | 83(1) |
| C(203) | 6496(4) | 7549(3) | 1630(3) | 60(1) |
| C(204) | 5559(4) | 7977(3) | 2000(3) | 75(1) |
| C(205) | 4778(5) | 8580(3) | 1548(3) | 95(2) |
| C(206) | 5004(5) | 8763(3) | 717(4) | 100(2) |
| C(207) | 6218(6) | 8650(5) | −626(4) | 189(3) |
| C(208) | 6935(4) | 6146(2) | 2808(2) | 51(1) |
| C(209) | 7747(3) | 5532(2) | 3270(2) | 47(1) |
| C(210) | 7310(5) | 4754(3) | 3989(3) | 67(1) |
| C(211) | 5391(4) | 5100(3) | 3830(3) | 71(1) |
| C(212) | 5730(4) | 5884(3) | 3123(3) | 65(1) |
| C(213) | 9951(4) | 5046(3) | 1815(2) | 62(1) |
| C(214) | 10699(6) | 4261(3) | 879(3) | 101(2) |
| C(215) | 10001(8) | 3526(5) | 1074(4) | 176(3) |
| C(216) | 12008(7) | 4115(7) | 470(5) | 245(5) |
| N(201) | 7290(3) | 6908(2) | 2108(2) | 67(1) |
| N(202) | 6154(4) | 4532(2) | 4276(2) | 72(1) |
| N(203) | 10011(3) | 4997(2) | 2596(2) | 61(1) |
| N(204) | 10627(4) | 4390(2) | 1632(2) | 91(1) |
| O(201) | 9538(2) | 6592(2) | 2392(2) | 57(1) |
| O(202) | 9616(2) | 5334(2) | 3808(2) | 59(1) |
| O(203) | 9348(2) | 5635(2) | 1382(2) | 79(1) |
| C(301) | 23(4) | 1000(3) | 2139(2) | 65(1) |
| C(302) | 799(4) | 908(3) | 2616(2) | 59(1) |
| C(303) | 368(3) | 1050(2) | 3352(2) | 46(1) |
| C(304) | −833(4) | 1308(3) | 3614(2) | 59(1) |
| C(305) | −1592(4) | 1393(3) | 3127(3) | 74(1) |
| C(306) | −1173(5) | 1239(3) | 2410(3) | 75(1) |
| C(307) | 484(4) | 850(3) | 1332(3) | 100(2) |
| C(308) | 1960(3) | 277(2) | 4112(2) | 47(1) |
| C(309) | 2761(3) | 279(2) | 4561(2) | 45(1) |
| C(310) | 3616(3) | −415(2) | 4748(2) | 51(1) |
| C(311) | 2907(4) | −1140(2) | 4164(2) | 60(1) |
| C(312) | 2053(4) | −490(2) | 3950(2) | 57(1) |
| C(313) | 4274(4) | 2171(3) | 3624(3) | 59(1) |
| C(314) | 5639(5) | 3323(3) | 2414(3) | 106(2) |
| C(315) | 5416(7) | 4076(4) | 1585(3) | 190(4) |
| C(316) | 6359(6) | 3611(4) | 2790(4) | 153(3) |
| N(301) | 1142(3) | 959(2) | 3861(2) | 52(1) |
| N(302) | 3713(3) | −1103(2) | 4535(2) | 54(1) |
| N(303) | 3083(3) | 2025(2) | 4146(2) | 54(1) |
| N(304) | 4445(3) | 3021(2) | 3009(2) | 77(1) |
| O(301) | 1412(2) | 1221(2) | 5330(1) | 59(1) |
| O(302) | 3494(2) | 773(2) | 5476(1) | 60(1) |
| O(303) | 5055(3) | 1567(2) | 3719(2) | 76(1) |

TABLE 3
Bond lengths (Å)

| | | | |
|---|---|---|---|
| C(1)-C(6) | 1.381(5) | C(208)-H(201) | 1.332(4) |
| C(1)-C(2) | 1.384(5) | C(208)-C(212) | 1.408(5) |
| C(1)-C(7) | 1.510(5) | C(208)-C(209) | 1.421(5) |
| C(2)-C(3) | 1.376(5) | C(209)-C(210) | 1.382(5) |
| C(2)-H(2) | 0.9300 | C(210)-N(202) | 1.336(5) |
| C(3)-C(4) | 1.383(4) | C(210)-H(210) | 0.9300 |
| C(3)-N(1) | 1.428(4) | C(211)-N(202) | 1.340(5) |
| C(4)-C(5) | 1.382(5) | C(211)-C(212) | 1.356(5) |
| C(4)-H(4) | 0.9300 | C(211)-N(211) | 0.9300 |
| C(5)-C(6) | 1.357(5) | C(212)-H(212) | 0.9300 |
| C(5)-H(5) | 0.9300 | C(213)-O(203) | 1.215(4) |
| C(6)-H(6) | 0.9300 | C(213)-N(204) | 1.331(5) |
| C(7)-H(7A) | 0.9600 | C(213)-N(203) | 1.404(4) |
| C(7)-H(7B) | 0.9600 | C(214)-C(215) | 1.440(7) |
| C(7)-H(7C) | 0.9600 | C(214)-N(204) | 1.452(5) |
| C(8)-N(1) | 1.344(4) | C(214)-C(216) | 1.499(8) |
| C(8)-C(12) | 1.403(4) | C(214)-H(214) | 0.9800 |
| C(8)-C(9) | 1.431(4) | C(215)-H(21A) | 0.9600 |
| C(9)-C(10) | 1.372(5) | C(215)-H(21B) | 0.9600 |
| C(9)-S(1) | 1.745(3) | C(215)-H(21C) | 0.9600 |
| C(10)-N(2) | 1.336(4) | C(216)-H(21D) | 0.9600 |
| C(10)-H(10) | 0.9300 | C(216)-H(21E) | 0.9600 |
| C(11)-C(12) | 1.347(4) | C(212)-H(21F) | 0.9600 |
| C(11)-N(2) | 1.344(4) | N(201)-H(201) | 0.8600 |
| C(11)-H(11) | 0.9300 | C(203)-H(203) | 0.8600 |
| C(12)-H(12) | 0.9300 | C(204)-H(20D) | 0.8600 |
| C(13)-O(3) | 1.215(4) | C(301)-C(306) | 1.375(6) |
| C(13)-N(4) | 1.336(4) | C(301)-C(302) | 1.388(5) |
| C(13)-N(3) | 1.400(4) | C(301)-C(307) | 1.507(5) |
| C(14)-N(4) | 1.455(5) | C(302)-C(303) | 1.380(4) |
| C(14)-C(15) | 1.475(7) | C(302)-H(302) | 0.9300 |
| C(14)-C(16) | 1.506(7) | C(303)-C(304) | 1.385(5) |
| C(14)-H(14) | 0.9800 | C(303)-N(301) | 1.432(4) |
| C(15)-H(15A) | 0.9600 | C(304)-C(305) | 1.389(5) |
| C(15)-H(15B) | 0.9600 | C(304)-H(304) | 0.9300 |
| C(15)-H(15C) | 0.9600 | C(305)-C(306) | 1.358(6) |
| C(16)-H(16A) | 0.9600 | C(305)-H(305) | 0.9300 |
| C(16)-H(16B) | 0.9600 | C(306)-H(306) | 0.9300 |
| C(16)-H(16C) | 0.9600 | C(307)-H(30A) | 0.9600 |
| N(1)-H(1) | 0.8600 | C(307)-H(30B) | 0.9600 |
| N(3)-S(1) | 1.620(3) | C(307)-H(30C) | 0.9600 |
| N(3)-H(3) | 0.8600 | C(307)-N(301) | 1.349(4) |
| N(4)-H(4A) | 0.8600 | C(308)-C(312) | 1.415(4) |
| O(1)-S(1) | 1.432(3) | C(308)-C(309) | 1.424(4) |
| O(2)-S(1) | 1.434(2) | C(309)-C(310) | 1.373(4) |
| S(2)-O(201) | 1.430(2) | C(310)-N(302) | 1.342(4) |
| S(2)-O(202) | 1.430(2) | C(310)-H(310) | 0.9300 |
| S(2)-N(203) | 1.602(3) | C(311)-C(312) | 1.337(5) |
| S(2)-C(209) | 1.767(4) | C(311)-N(302) | 1.345(4) |
| S(3)-O(302) | 1.422(2) | C(311)-H(311) | 0.9300 |
| S(3)-O(301) | 1.427(2) | C(312)-H(312) | 0.9300 |
| S(3)-N(303) | 1.619(3) | C(313)-O(303) | 1.217(4) |
| S(3)-C(309) | 1.756(3) | C(313)-N(304) | 1.343(5) |
| C(201)-C(206) | 1.370(7) | C(313)-N(303) | 1.400(5) |
| C(201)-C(202) | 1.406(6) | C(314)-N(304) | 1.469(6) |
| C(201)-C(207) | 1.486(6) | C(314)-C(316) | 1.489(8) |
| C(202)-C(203) | 1.376(5) | C(314)-C(315) | 1.520(7) |
| C(202)-H(202) | 0.9300 | C(314)-H(314) | 0.9800 |
| C(203)-C(204) | 1.356(5) | C(315)-H(31A) | 0.9600 |
| C(203)-N(201) | 1.436(4) | C(315)-H(31B) | 0.9600 |
| C(204)-C(205) | 1.380(6) | C(315)-H(31C) | 0.9600 |
| C(204)-H(204) | 0.9300 | C(316)-H(31D) | 0.9600 |
| C(205)-C(206) | 1.336(6) | C(316)-H(31E) | 0.9600 |
| C(205)-H(205) | 0.9300 | C(316)-H(31F) | 0.9600 |
| C(206)-H(206) | 0.9300 | N(301)-H(301) | 0.8600 |
| C(207)-H(20A) | 0.9600 | N(303)-H(303) | 0.8600 |
| C(207)-H(20B) | 0.9600 | N(304)-H(30D) | 0.8600 |
| C(207)-H(20C) | 0.9600 | | |

TABLE 4
Bond Angles (°)

| | | | |
|---|---|---|---|
| C(6)-C(1)-C(2) | 117.8(4) | C(212)-C(208)-C(209) | 115.6(3) |
| C(6)-C(1)-C(7) | 122.6(4) | C(210)-C(209)-C(208) | 119.1(4) |
| C(2)-C(1)-C(7) | 119.7(3) | C(210)-C(209)-S(2) | 115.5(3) |
| C(3)-C(2)-C(1) | 120.7(3) | C(208)-C(209)-S(2) | 125.4(3) |
| C(3)-C(2)-H(2) | 119.6 | N(202)-C(210)-C(209) | 123.5(4) |
| C(1)-C(2)-H(2) | 119.6 | N(202)-C(210)-H(210) | 118.2 |
| C(2)-C(3)-C(4) | 120.6(3) | C(209)-C(210)-H(210) | 118.2 |
| C(2)-C(3)-N(1) | 121.5(3) | N(202)-C(211)-C(212) | 123.3(4) |
| C(4)-C(3)-N(1) | 117.7(3) | N(202)-C(211)-H(211) | 118.4 |
| C(3)-C(4)-C(5) | 118.4(4) | C(212)-C(211)-H(211) | 118.4 |
| C(3)-C(4)-H(4) | 120.7 | C(211)-C(212)-C(208) | 120.9(4) |
| C(5)-C(4)-H(4) | 120.7 | C(211)-C(212)-H(212) | 119.6 |
| C(6)-C(5)-C(4) | 120.3(3) | C(208)-C(212)-H(212) | 119.6 |
| C(6)-C(5)-H(5) | 119.8 | O(203)-C(213)-N(204) | 126.6(3) |
| C(4)-C(5)-H(5) | 119.8 | O(203)-C(213)-N(203) | 121.6(3) |
| C(5)-C(6)-C(1) | 122.0(4) | N(204)-C(213)-N(203) | 111.8(3) |
| C(5)-C(6)-H(6) | 119.0 | C(215)-C(214)-N(204) | 113.2(5) |
| C(1)-C(6)-H(6) | 119.0 | C(215)-C(214)-C(216) | 111.0(6) |
| C(1)-C(7)-H(7A) | 109.5 | N(204)-C(214)-C(216) | 107.8(5) |
| C(1)-C(7)-H(7B) | 109.5 | C(215)-C(214)-H(214) | 108.2 |
| H(7A)-C(7)-H(7B) | 109.5 | N(204)-C(214)-H(214) | 108.2 |
| C(1)-C(7)-H(7C) | 109.5 | C(216)-C(214)-H(214) | 108.2 |
| H(7A)-C(7)-H(7C) | 109.5 | C(214)-C(215)-H(21A) | 109.5 |
| H(7B)-C(7)-H(7C) | 109.5 | C(214)-C(215)-H(21B) | 109.5 |
| N(1)-C(8)-C(12) | 122.7(3) | H(21A)-C(215)-H(21B) | 109.5 |
| N(1)-C(8)-C(9) | 121.3(3) | C(214)-C(215)-H(21C) | 109.5 |
| C(12)-C(8)-C(9) | 116.0(3) | H(21A)-C(215)-H(21C) | 109.5 |
| C(10)-C(9)-C(8) | 118.7(3) | H(21B)-C(215)-H(21C) | 109.5 |
| C(8)-C(9)-S(1) | 124.6(3) | C(214)-C(216)-H(21E) | 109.5 |
| N(2)-C(10)-C(9) | 123.0(3) | H(21D)-C(216)-H(21E) | 109.5 |
| N(2)-C(10)-H(10) | 118.5 | C(214)-C(216)-H(21F) | 109.5 |
| C(9)-C(10)-H(10) | 118.5 | H(21D)-C(216)-H(21F) | 109.5 |
| C(12)-C(11)-N(2) | 122.2(3) | H(21E)-C(216)-H(21F) | 109.5 |
| C(12)-C(11)-H(11) | 118.9 | C(208)-N(201)-C(203) | 124.2(3) |
| N(2)-C(11)-H(11) | 118.9 | C(208)-N(201)-H(201) | 117.9 |
| C(11)-C(12)-C(8) | 121.3(3) | C(203)-N(201)-H(201) | 117.9 |
| C(11)-C(12)-H(12) | 119.4 | C(210)-N(202)-C(211) | 117.6(4) |
| C(8)-C(12)-H(12) | 119.4 | C(213)-N(203)-S(2) | 125.1(3) |
| O(3)-C(13)-N(4) | 124.8(3) | C(213)-N(203)-H(203) | 117.5 |
| O(3)-C(13)-N(3) | 123.3(3) | S(2)-N(203)-H(203) | 117.5 |
| N(4)-C(13)-N(3) | 111.9(3) | C(213)-N(204)-C(214) | 123.5(4) |
| N(4)-C(14)-C(15) | 111.9(4) | C(213)-N(204)-H(20D) | 118.3 |
| N(4)-C(14)-C(16) | 108.2(4) | C(214)-N(204)-H(20D) | 118.3 |
| C(15)-C(14)-C(16) | 113.5(5) | C(306)-C(301)-C(302) | 118.9(4) |
| N(4)-C(14)-H(14) | 107.7 | C(306)-C(301)-C(307) | 120.4(4) |
| C(15)-C(14)-H(14) | 107.7 | C(302)-C(301)-C(307) | 120.7(4) |
| C(16)-C(14)-H(14) | 107.7 | C(303)-C(302)-C(301) | 120.2(4) |
| C(14)-C(15)-H(15A) | 109.5 | C(303)-C(302)-H(302) | 119.9 |
| C(14)-C(15)-H(15B) | 109.5 | C(301)-C(302)-H(302) | 119.9 |
| H(15A)-C(15)-H(15B) | 109.5 | C(302)-C(303)-C(304) | 120.7(3) |
| C(14)-C(15)-H(15C) | 109.5 | C(302)-C(303)-N(301) | 121.7(3) |
| H(15A)-C(15)-H(15C) | 109.5 | C(304)-C(303)-N(301) | 117.6(3) |
| H(15B)-C(15)-H(15C) | 109.5 | C(303)-C(304)-C(305) | 118.0(4) |
| C(14)-C(16)-H(16A) | 109.5 | C(303)-C(304)-H(304) | 121.0 |
| C(14)-C(16)-H(16B) | 109.5 | C(305)-C(304)-H(304) | 121.0 |
| H(16A)-C(16)-H(16B) | 109.5 | C(306)-C(305)-C(304) | 121.4(4) |
| C(14)-C(16)-H(16C) | 109.5 | C(306)-C(305)-H(305) | 119.3 |
| H(16A)-C(16)-H(16C) | 109.5 | C(304)-C(305)-H(305) | 119.3 |
| H(16B)-C(16)-H(16C) | 109.5 | C(305)-C(306)-C(301) | 120.8(4) |
| C(8)-N(1)-C(3) | 126.3(3) | C(305)-C(306)-H(306) | 119.6 |
| C(8)-N(1)-H(1) | 116.8 | C(301)-C(306)-H(306) | 119.6 |
| C(10)-N(2)-C(11) | 118.8(3) | C(301)-C(307)-H(30B) | 109.5 |
| C(13)-N(3)-S(1) | 127.8(3) | H(30A)-C(307)-H(30B) | 109.5 |
| C(13)-N(3)-H(3) | 116.1 | C(301)-C(307)-H(30C) | 109.5 |
| S(1)-N(3)-H(3) | 116.1 | H(30A)-C(307)-H(30C) | 109.5 |
| C(13)-N(4)-C(14) | 123.0(3) | H(30B)-C(307)-H(30C) | 109.5 |
| C(13)-N(4)-H(4A) | 118.5 | N(301)-C(308)-C(312) | 121.1(3) |
| C(14)-N(4)-H(4A) | 118.5 | N(301)-C(308)-C(309) | 122.5(3) |
| O(1)-S(1)-O(2) | 120.30(16) | C(312)-C(308)-C(309) | 116.3(3) |
| O(1)-S(1)-N(3) | 108.69(15) | C(310)-C(309)-C(308) | 118.9(3) |
| O(2)-S(1)-N(3) | 105.71(16) | C(310)-C(309)-S(3) | 116.7(3) |
| O(1)-S(1)-C(9) | 107.99(16) | C(308)-C(309)-S(3) | 124.2(3) |
| O(2)-S(1)-C(9) | 106.71(16) | N(302)-C(310)-C(309) | 122.3(3) |
| N(3)-S(1)-C(9) | 106.70(15) | N(302)-C(310)-H(310) | 118.8 |
| O(201)-S(2)-O(202) | 120.20(15) | C(309)-C(310)-H(310) | 118.8 |
| O(201)-S(2)-N(203) | 110.18(15) | C(312)-C(311)-N(302) | 122.6(3) |
| O(202)-S(2)-N(203) | 104.32(14) | C(312)-C(311)-H(311) | 118.7 |
| O(201)-S(2)-C(209) | 107.37(16) | N(302)-C(311)-H(311) | 118.7 |
| O(202)-S(2)-O(209) | 107.04(16) | C(311)-C(312)-C(308) | 120.5(3) |
| N(203)-S(2)-C(209) | 107.04(16) | C(311)-C(312)-H(312) | 119.7 |
| O(302)-S(3)-O(301) | 120.54(15) | C(308)-C(312)-H(312) | 119.7 |
| O(302)-S(3)-N(303) | 109.44(16) | O(303)-C(313)-N(304) | 124.4(4) |
| O(301)-S(3)-N(303) | 105.01(16) | O(303)-C(313)-N(303) | 122.0(4) |
| O(302)-S(3)-C(309) | 107.22(15) | N(304)-C(313)-N(303) | 113.5(4) |
| O(301)-S(3)-C(309) | 106.83(16) | N(304)-C(314)-C(316) | 111.9(4) |
| N(303)-S(3)-C(309) | 107.12(15) | N(304)-C(314)-C(315) | 106.5(5) |
| C(206)-C(201)-C(202) | 119.1(4) | C(316)-C(314)-C(315) | 112.7(5) |
| C(206)-C(201)-C(207) | 122.0(5) | N(304)-C(314)-H(314) | 108.5 |
| C(202)-C(201)-C(207) | 118.9(6) | C(316)-C(314)-H(314) | 108.5 |
| C(203)-C(202)-C(201) | 117.8(4) | C(315)-C(314)-H(314) | 108.5 |
| C(203)-C(202)-H(202) | 121.1 | C(314)-C(315)-H(31A) | 109.5 |
| C(201)-C(202)-H(202) | 121.1 | C(314)-C(315)-H(31B) | 109.5 |
| C(204)-C(203)-C(202) | 120.8(4) | H(31A)-C(315)-H(31B) | 109.5 |
| C(204)-C(203)-N(201) | 121.2(4) | C(314)-C(315)-H(31C) | 109.5 |
| C(202)-C(203)-N(201) | 118.0(4) | H(31A)-C(315)-H(31C) | 109.5 |
| C(203)-C(204)-C(205) | 121.4(4) | H(31B)-C(315)-H(31C) | 109.5 |
| C(203)-C(204)-H(204) | 119.3 | C(314)-C(316)-H(31D) | 109.5 |
| C(205)-C(204)-H(204) | 119.3 | C(314)-C(316)-H(31E) | 109.5 |
| C(206)-C(205)-C(204) | 118.0(5) | H(31D)-C(316)-H(31E) | 109.5 |
| C(206)-C(205)-H(205) | 121.0 | C(314)-C(316)-H(31F) | 109.5 |
| C(204)-C(205)-H(205) | 121.0 | H(31D)-C(316)-H(31F) | 109.5 |
| C(205)-C(206)-C(201) | 122.8(5) | H(31E)-C(316)-H(31F) | 109.5 |
| C(205)-C(206)-H(206) | 118.6 | C(308)-N(301)-C(303) | 126.5(3) |
| C(201)-C(206)-H(206) | 118.6 | C(308)-N(301)-H(301) | 116.8 |
| C(201)-C(207)-H(20A) | 109.5 | C(303)-N(301)-H(301) | 116.8 |
| C(201)-C(207)-H(20B) | 109.5 | C(310)-N(302)-C(311) | 119.0(3) |
| H(20A)-C(207)H(20B) | 109.5 | C(313)-N(303)-S(3) | 122.9(3) |
| C(201)-C(207)-H(20C) | 109.5 | C(313)-N(303)-H(303) | 118.5 |
| H(20A)-C(207)H(20C) | 109.5 | S(3)-N(303)-H(303) | 118.5 |
| H(20B)-C(207)- | 109.5 | C(313)-N(304)-C(314) | 122.1(4) |
| H(20C) | | | |
| N(201)-C(208)-C(212) | 121.8(3) | C(313)-N(304)-H(30D) | 118.9 |
| N(201)-C(208)-C(209) | 122.6(4) | C(314)-N(304)-H(30D) | 118.9 |

TABLE 5
Anisotropic displacement parameters (Å² × 10³)

| | U₁₁ | U₂₂ | U₃₃ | U₂₃ | U₁₃ | U₁₂ |
|---|---|---|---|---|---|---|
| Cl(1) | 49(1) | 42(1) | 85(1) | −19(1) | −19(1) | 0(1) |
| Cl(2) | 117(1) | 49(1) | 101(1) | −35(1) | −75(1) | 25(1) |
| Cl(3) | 64(1) | 52(1) | 62(1) | −16(1) | −10(1) | 3(1) |
| C(1) | 56(3) | 52(3) | 63(2) | −19(2) | −24(2) | 4(2) |
| C(2) | 60(3) | 50(2) | 48(2) | −16(2) | −22(2) | 1(2) |
| C(3) | 55(3) | 38(2) | 46(2) | −11(2) | −22(2) | −3(2) |
| C(4) | 69(3) | 68(3) | 50(2) | −26(2) | −19(2) | −1(2) |
| C(5) | 87(4) | 81(3) | 44(2) | −21(2) | −20(2) | −7(3) |
| C(6) | 85(4) | 58(3) | 60(3) | −9(2) | −39(2) | −4(3) |
| C(7) | 82(4) | 96(4) | 103(3) | −40(3) | −44(3) | 40(3) |
| C(8) | 42(2) | 40(2) | 47(2) | −24(2) | −9(2) | −4(2) |
| C(9) | 36(2) | 45(2) | 45(2) | −21(2) | −21(2) | 6(2) |
| C(10) | 55(3) | 54(2) | 48(2) | −26(2) | −25(2) | 8(2) |
| C(11) | 46(2) | 46(2) | 45(2) | −17(2) | −10(2) | −7(2) |
| C(12) | 39(2) | 40(2) | 49(2) | −15(2) | −16(2) | −2(2) |
| C(13) | 42(2) | 53(3) | 54(2) | −28(2) | −7(2) | −4(2) |
| C(14) | 126(5) | 73(3) | 55(3) | −33(2) | −3(3) | −2(3) |
| C(15) | 165(7) | 243(9) | 83(4) | −62(5) | −33(4) | −59(6) |
| C(16) | 192(7) | 190(7) | 92(4) | −86(4) | 0(4) | 48(6) |
| N(1) | 39(2) | 44(2) | 49(2) | −14(2) | −9(1) | −6(2) |
| N(2) | 48(2) | 58(2) | 53(2) | −26(2) | −16(2) | −2(2) |
| N(3) | 53(2) | 53(2) | 55(2) | −29(2) | −17(2) | 6(2) |
| N(4) | 81(3) | 55(2) | 51(2) | −22(2) | −10(2) | 7(2) |
| O(1) | 60(2) | 62(2) | 87(2) | −43(2) | −15(1) | −15(2) |
| O(2) | 56(2) | 101(2) | 68(2) | −38(2) | −36(1) | 5(2) |
| O(3) | 62(2) | 51(2) | 63(2) | −22(1) | −13(1) | 1(2) |
| S(1) | 47(1) | 66(1) | 61(1) | −35(1) | −19(1) | −5(1) |
| S(2) | 60(1) | 44(1) | 58(1) | −25(1) | −29(1) | 7(1) |
| S(3) | 64(1) | 45(1) | 55(1) | −28(1) | −28(1) | 7(1) |
| C(201) | 84(4) | 125(5) | 53(3) | 11(3) | −40(3) | −23(4) |
| C(202) | 73(3) | 88(4) | 72(3) | −15(3) | −28(3) | −6(3) |
| C(203) | 50(3) | 51(3) | 65(3) | −5(2) | −26(2) | −8(2) |
| C(204) | 75(3) | 61(3) | 81(3) | −16(2) | −36(3) | 4(3) |
| C(205) | 96(4) | 81(4) | 94(4) | −17(3) | −40(3) | −5(3) |
| C(206) | 60(4) | 79(4) | 123(4) | 0(3) | −40(3) | −9(3) |
| C(207) | 140(6) | 264(9) | 112(5) | −27(5) | −59(4) | 5(6) |
| C(208) | 52(3) | 44(2) | 58(2) | −16(2) | −27(2) | 4(2) |
| C(209) | 57(3) | 36(2) | 55(2) | −18(2) | −24(2) | −2(2) |
| C(210) | 94(4) | 44(3) | 71(3) | −19(2) | −36(3) | −6(2) |
| C(211) | 65(3) | 61(3) | 86(3) | −23(3) | −24(3) | −19(3) |
| C(212) | 56(3) | 55(3) | 79(3) | −17(2) | −30(2) | −4(2) |
| C(213) | 89(3) | 48(3) | 50(2) | −18(2) | −26(2) | 1(2) |
| C(214) | 169(6) | 77(4) | 64(3) | −42(3) | −31(3) | 13(4) |
| C(215) | 303(11) | 137(6) | 106(5) | −49(4) | −35(5) | −95(7) |
| C(216) | 192(9) | 422(15) | 209(8) | −253(10) | 37(7) | −57(9) |
| N(201) | 56(2) | 56(2) | 66(2) | 2(2) | −29(2) | −5(2) |
| N(202) | 81(3) | 52(2) | 80(2) | −13(2) | −31(2) | −20(2) |
| N(203) | 83(3) | 51(2) | 62(2) | −32(2) | −42(2) | 29(2) |
| N(204) | 148(4) | 71(3) | 65(2) | −41(2) | −52(2) | 47(3) |
| O(201) | 63(2) | 40(2) | 64(2) | −17(1) | −22(1) | −3(1) |
| O(202) | 72(2) | 63(2) | 60(2) | −32(1) | −40(1) | 12(1) |
| O(203) | 118(3) | 61(2) | 67(2) | −24(2) | −56(2) | 20(2) |
| C(301) | 73(3) | 66(3) | 63(3) | −23(2) | −31(2) | −12(2) |
| C(302) | 64(3) | 57(3) | 65(2) | −28(2) | −25(2) | −3(2) |
| C(303) | 50(3) | 41(2) | 54(2) | −19(2) | −28(2) | 2(2) |
| C(304) | 54(3) | 54(3) | 61(2) | −18(2) | −18(2) | 1(2) |
| C(305) | 47(3) | 71(3) | 91(3) | −19(3) | −28(2) | −3(2) |
| C(306) | 85(4) | 73(3) | 77(3) | −17(3) | −45(3) | −21(3) |
| C(307) | 122(5) | 123(4) | 74(3) | −44(3) | −33(3) | −36(4) |
| C(308) | 55(3) | 44(2) | 47(2) | −21(2) | −20(2) | 1(2) |
| C(309) | 54(2) | 38(2) | 52(2) | −23(2) | −23(2) | 6(2) |
| C(310) | 62(3) | 45(2) | 51(2) | −20(2) | −23(2) | −2(2) |
| C(311) | 76(3) | 43(2) | 78(3) | −35(2) | −36(2) | 10(2) |
| C(312) | 71(3) | 49(3) | 72(3) | −34(2) | −40(2) | 8(2) |
| C(313) | 72(3) | 47(3) | 69(3) | −35(2) | −22(2) | 8(2) |
| C(314) | 107(5) | 64(3) | 94(4) | −33(3) | 37(3) | −4(3) |
| C(315) | 265(9) | 114(5) | 82(4) | −15(4) | 45(5) | 2(6) |
| C(316) | 119(6) | 149(6) | 207(7) | −115(6) | 27(5) | −60(5) |
| N(301) | 66(2) | 42(2) | 67(2) | −31(2) | −39(2) | 14(2) |
| N(302) | 62(2) | 45(2) | 68(2) | −31(2) | −28(2) | 3(2) |
| N(303) | 60(2) | 37(2) | 65(2) | −24(2) | −23(2) | 9(2) |
| N(304) | 90(3) | 51(2) | 73(2) | −25(2) | −8(2) | 12(2) |
| O(301) | 58(2) | 61(2) | 64(2) | −37(1) | −17(1) | 10(1) |
| O(302) | 80(2) | 57(2) | 64(2) | −30(1) | −47(2) | 12(2) |
| O(303) | 71(2) | 53(2) | 97(2) | −36(2) | −18(2) | 14(2) |

TABLE 6
Hydrogen coordinates (×10⁴) and isotropic displacement
parameters (Å² × 10³)

| | x | y | z | U |
|---|---|---|---|---|
| H(2) | −2445 | 9265 | 3017 | 64 |
| H(4) | −289 | 8743 | 1053 | 74 |
| H(5) | −1522 | 9703 | 195 | 86 |
| H(6) | −3184 | 10414 | 743 | 83 |
| H(7A) | −4134 | 11007 | 1945 | 144 |
| H(7B) | −4099 | 10288 | 2875 | 144 |
| H(7C) | −4832 | 10114 | 2378 | 144 |
| H(10) | 345 | 6190 | 4982 | 59 |
| H(11) | −3036 | 6581 | 4939 | 56 |
| H(12) | −2575 | 7713 | 3602 | 52 |
| H(14) | 3159 | 7843 | 248 | 106 |
| H(15A) | 1198 | 7467 | 551 | 240 |
| H(15B) | 1689 | 6535 | 528 | 240 |
| H(15C) | 2016 | 7435 | −311 | 240 |
| H(16A) | 4693 | 6697 | 424 | 249 |
| H(16B) | 4284 | 6952 | −402 | 249 |
| H(16C) | 3838 | 6068 | 430 | 249 |
| H(1) | 295 | 8455 | 2395 | 56 |
| H(3) | 2429 | 6406 | 2919 | 62 |
| H(4A) | 2950 | 6339 | 1746 | 79 |
| H(202) | 7376 | 7444 | 517 | 100 |
| H(204) | 5439 | 7861 | 2572 | 90 |
| H(205) | 4116 | 8850 | 1813 | 114 |
| H(206) | 4486 | 9172 | 406 | 119 |
| H(20A) | 7057 | 8731 | −905 | 283 |
| H(20B) | 5730 | 9128 | −885 | 283 |
| H(20C) | 6032 | 8106 | −689 | 283 |
| H(210) | 7849 | 4361 | 4289 | 81 |
| H(211) | 4588 | 4947 | 4015 | 85 |
| H(212) | 5159 | 6254 | 2842 | 78 |
| H(214) | 10392 | 4818 | 469 | 122 |
| H(21A) | 9166 | 3640 | 1326 | 264 |
| H(21B) | 10293 | 2973 | 1472 | 264 |
| H(21C) | 10078 | 3475 | 553 | 264 |
| H(21D) | 12445 | 4628 | 317 | 368 |
| H(21E) | 12082 | 4040 | −39 | 368 |
| H(21F) | 12332 | 3582 | 869 | 368 |
| H(201) | 8043 | 7024 | 1932 | 80 |
| H(203) | 10460 | 4569 | 2872 | 73 |
| H(20D) | 11043 | 4025 | 1978 | 109 |
| H(302) | 1612 | 749 | 2439 | 70 |
| H(304) | −1123 | 1420 | 4101 | 70 |
| H(305) | −2403 | 1559 | 3296 | 88 |
| H(306) | −1702 | 1296 | 2099 | 90 |
| H(30A) | 1065 | 349 | 1417 | 150 |
| H(30B) | 859 | 1384 | 860 | 150 |
| H(30C) | −179 | 720 | 1205 | 150 |
| H(310) | 4148 | −407 | 5031 | 61 |
| H(311) | 2946 | −1638 | 4052 | 72 |
| H(312) | 1516 | −543 | 3693 | 68 |
| H(314) | 6081 | 2819 | 2290 | 127 |
| H(31A) | 4826 | 3897 | 1429 | 286 |
| H(31B) | 6158 | 4192 | 1132 | 286 |
| H(31C) | 5124 | 4614 | 1669 | 286 |
| H(31D) | 6443 | 3122 | 3324 | 230 |
| H(31E) | 5953 | 4122 | 2892 | 230 |
| H(31F) | 7145 | 3776 | 2395 | 230 |
| H(301) | 1078 | 1381 | 4023 | 63 |
| H(303) | 2549 | 2461 | 4025 | 64 |
| H(30D) | 3836 | 3401 | 2961 | 93 |

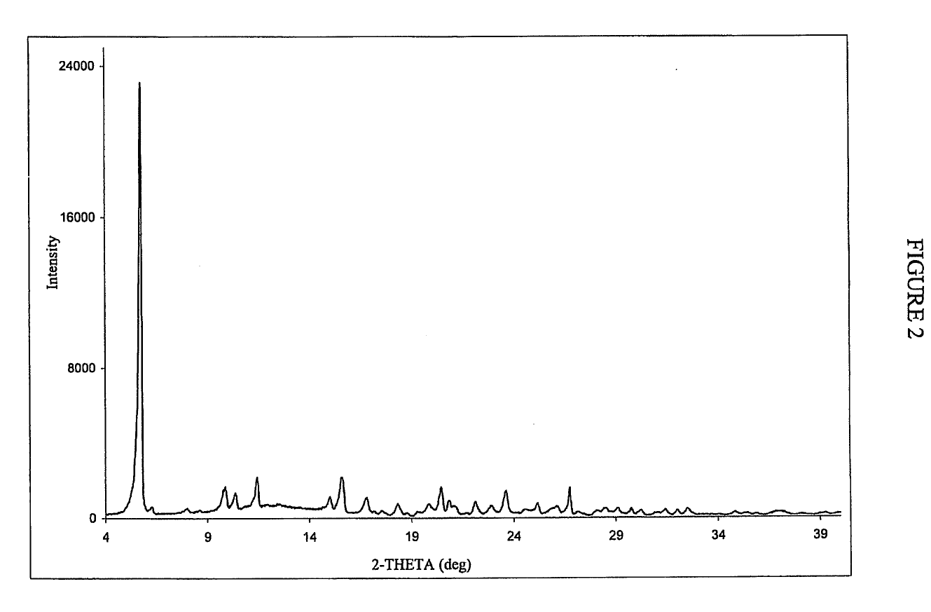

In one embodiment of the invention, Torsemide hydrochloride also gives distinctive x-ray powder diffraction pattern, as depicted in FIG. 2. In one embodiment of the invention, the pattern has a characteristic peak (expressed in degree 2θ±0.2) at about 5.8. In another embodiment, the peaks 5.8 and 11.6 are unique to torsemide hydrochloride. In another embodiment, the peaks 5.8, 11.6 and 15.8 are unique to torsemide hydrochloride.

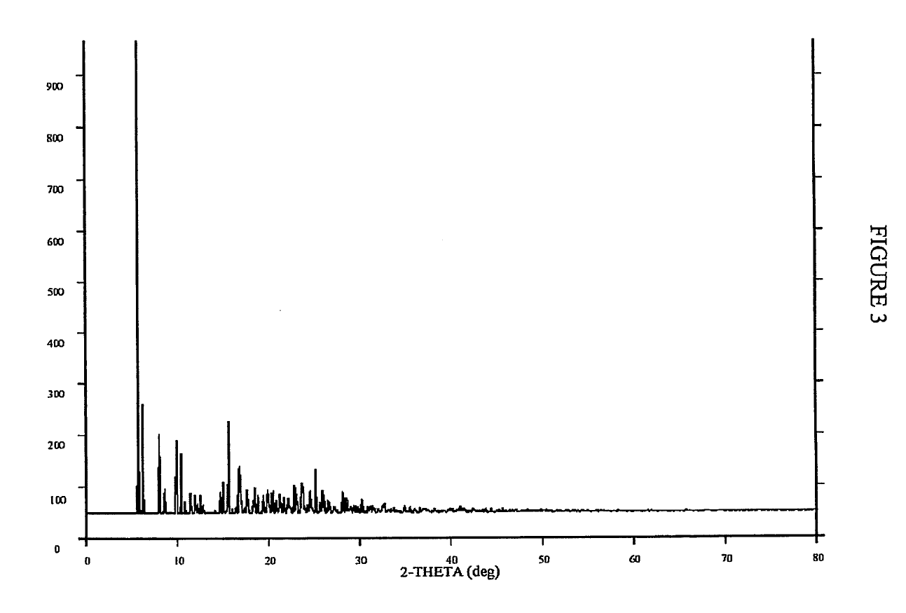

In one embodiment of the invention, the results of a single crystal x-ray analysis characterize one crystal placed in the x-ray beam. In another embodiment, crystallographic data on a large group of crystals provides powder x-ray diffraction In another embodiment, if the powder consists of a pure crystalline compound, a simple powder diagram is obtained. In one embodiment of the invention, a simple calculation can be done to convert the single crystal analysis to powder x-ray diagram to compare the results of a single crystal analysis and a powder x-ray analysis. In another embodiment, this conversion is possible because the single crystal experiment routinely determines the unit cell dimensions, space group, and atomic positions. These parameters provide a basis for calculating a perfect powder pattern. In another embodiment, comparing this calculated powder pattern and the powder pattern experimentally obtained from a large collection of crystals confirms if the results of the two techniques are the same. In another embodiment, the results for torsemide hydrochloride are graphically displayed in FIGS. 2 and 3 and in Table 7.

TABLE 7
Calculated from single crystal x-ray analysis powder diffraction
pattern (λ = 1.5418 Å radiation) where
in I/I₁ represents the relative intensity:

| 2θ (°) | I/I₁ | h | k | l |
|---|---|---|---|---|
| 5.696 | 1000 | 0 | 0 | 1 |
| 6.246 | 222 | 0 | 1 | 1 |
| 7.993 | 160 | 1 | 0 | 0 |
| 9.776 | 61 | 1 | 1 | 0 |
| 9.870 | 147 | 0 | 1 | −1 |
| 10.364 | 120 | 0 | 1 | 2 |
| 15.603 | 58 | 1 | −1 | 2 |
| 15.633 | 112 | 1 | 0 | −2 |
| 16.734 | 67 | 1 | 3 | 2 |
| 16.810 | 88 | 1 | −2 | 1 |
| 18.484 | 52 | 2 | 1 | 3 |

In one embodiment of the invention, the powder diffraction pattern for torsemide hydrochloride calculated from single crystal x-ray analysis is according to Table 7. In one embodiment of the invention, the experimentally derived x-ray powder diffraction pattern of torsemide hydrochloride is shown in FIG. 2. In another embodiment, FIG. 3 corresponds to the x-ray diffraction pattern derived from the single crystal x-ray data. In one embodiment of the invention, the peaks overlap. In another embodiment, the peak overlap indicates that the two techniques yield the same results. In one embodiment of the invention, the primary powder x-ray diffraction peaks provide an unambiguous description of the crystalline state of torsemide hydrochloride.

In one embodiment of the invention, pure crystalline organic compound has a definite melting point range. In one embodiment of the invention, melting point is the point at which the sample is entirely in the liquid phase. In one embodiment of the invention, the melting point range of crystalline torsemide hydrochloride is from 65 to 70° C. (dec.). In another embodiment, the melting point range is determined by the capillary method.

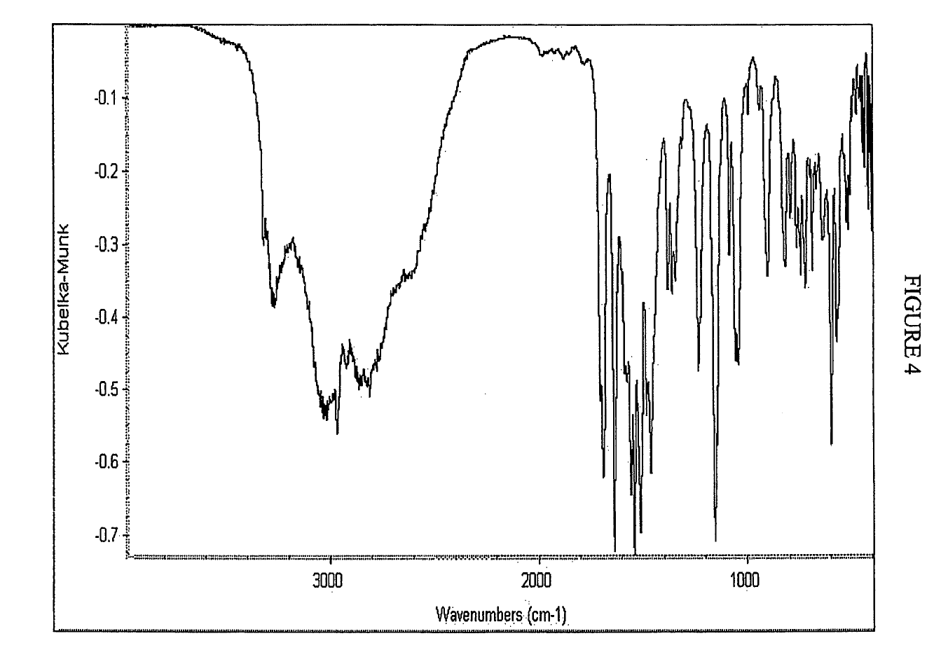

In one embodiment of the invention, the crystalline torsemide hydrochloride is characterized by an infrared absorption spectrum in potassium bromide as depicted in FIG. 4.

In one embodiment of the present invention, the crystalline form of torsemide hydrochloride is characterized by a powder x-ray diffraction pattern comprising the peak at about 6.0±0.3 degrees two-theta.

Furthermore, in one embodiment of the present invention, the crystalline form of torsemide hydrochloride is a crystalline form II of torsemide hydrochloride, characterized by a powder x-ray diffraction pattern substantially as represented in FIG. 2.

Furthermore, in one embodiment of the present invention, the crystalline form of torsemide hydrochloride is a crystalline form II of torsemide hydrochloride, characterized by a single crystal x-ray crystallographic analysis with crystal parameters that are approximately equal to the following:

| Space group | P-1 |
|---|---|
| Cell dimensions | |
| a (Å) | 11.691 |
| b (Å) | 16.649 |
| c (Å) | 18.130 |
| α (°) | 62.79 |
| β (°) | 71.25 |
| γ (°) | 79.44 |
| V (Å³) | 2969.1 |
| Z (molecules/units cell) | 6 |
| Density (g/cm³) | 1.288 |

wherein a, b and c represent the lengths of the sides of the unit cell; α, β and γ represent the relative angles of the cell sides; V represents the volume of the cell; and Z represents the number of molecules in the cell unit.

Furthermore, in one embodiment of the present invention, the crystalline form of torsemide hydrochloride is a crystalline form II of torsemide hydrochloride, characterized by an IR absorption spectrum substantially as represented in FIG. 4.

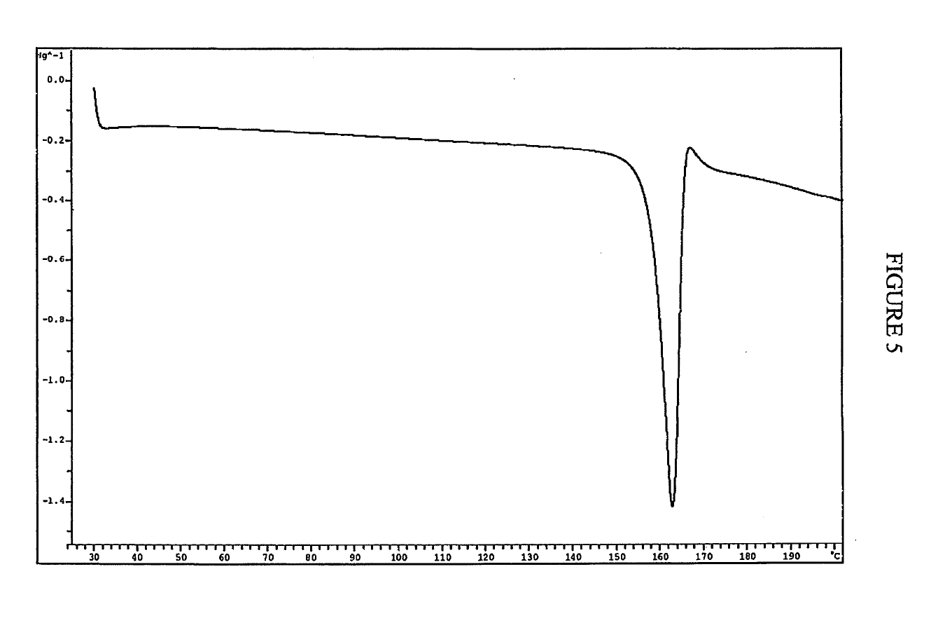

Furthermore, in one embodiment of the present invention, the crystalline form of torsemide hydrochloride is a crystalline form II of torsemide hydrochloride, characterized by a differential scanning calorimetric (DSC) thermogram substantially as represented in FIG. 5.

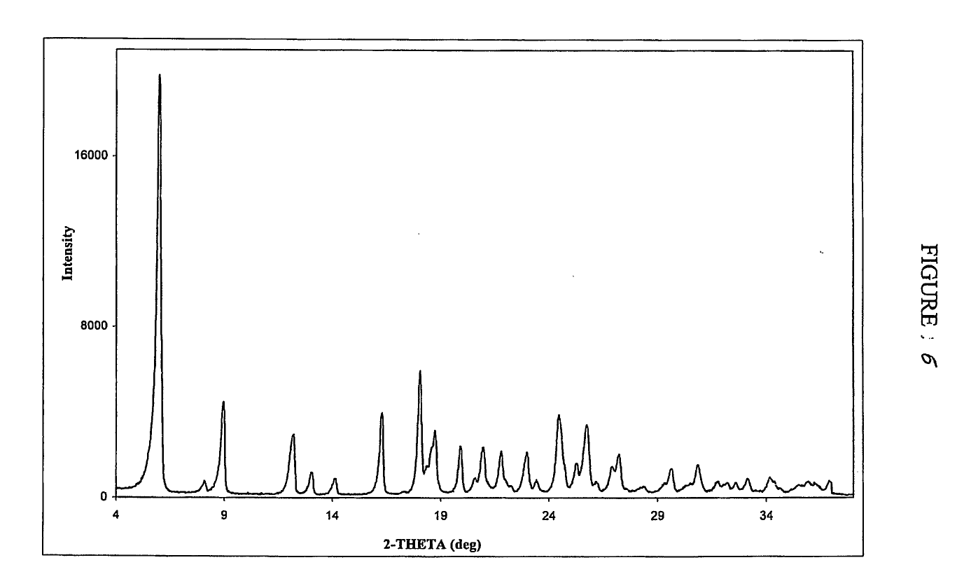

Furthermore, in one embodiment of the present invention, the crystalline form of torsemide hydrochloride is a crystalline form I of torsemide hydrochloride, characterized by a powder x-ray diffraction pattern substantially as represented in FIG. 6.

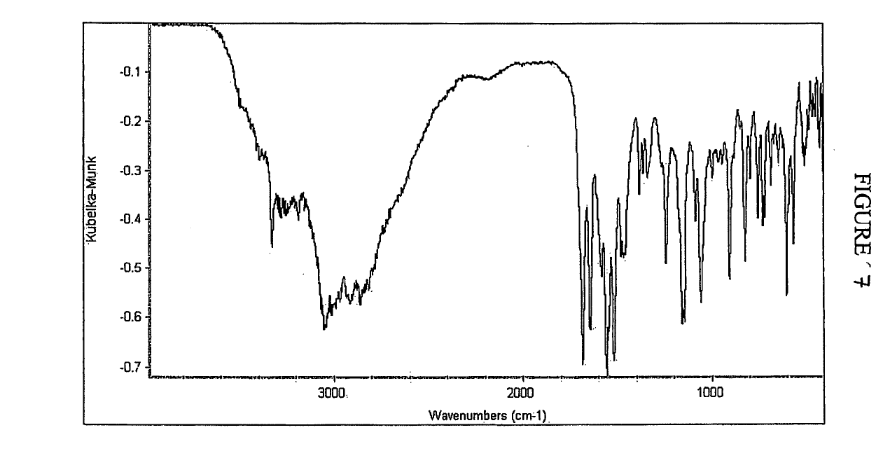

Furthermore, in one embodiment of the present invention, the crystalline form of torsemide hydrochloride is a crystalline form I of torsemide hydrochloride, characterized by an IR absorption spectrum substantially as represented in FIG. 7.

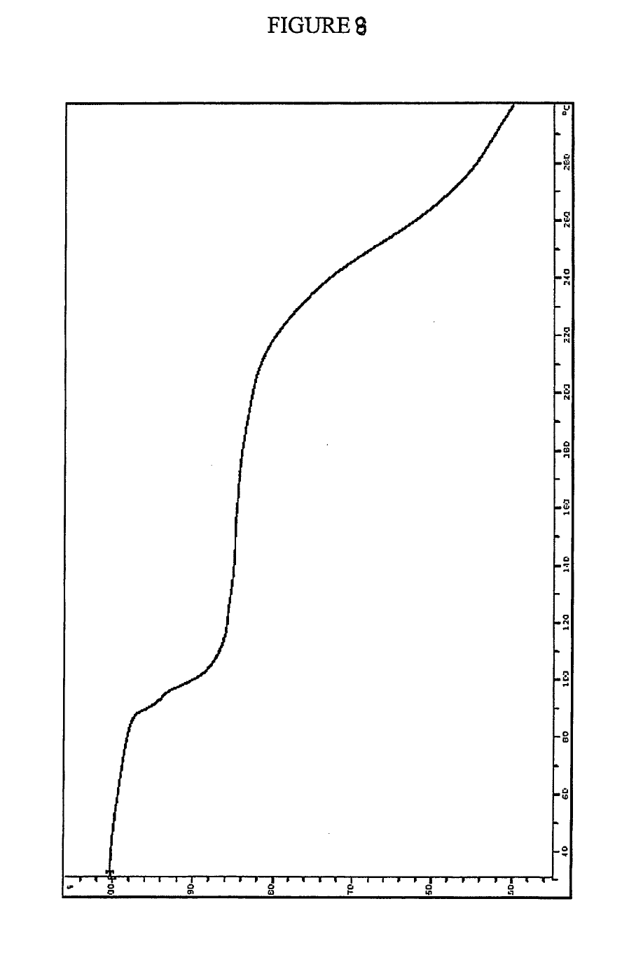

Furthermore, in one embodiment of the present invention, the crystalline form of torsemide hydrochloride is a crystalline form I of torsemide hydrochloride, characterized by the TGA as represented in FIG. 8.

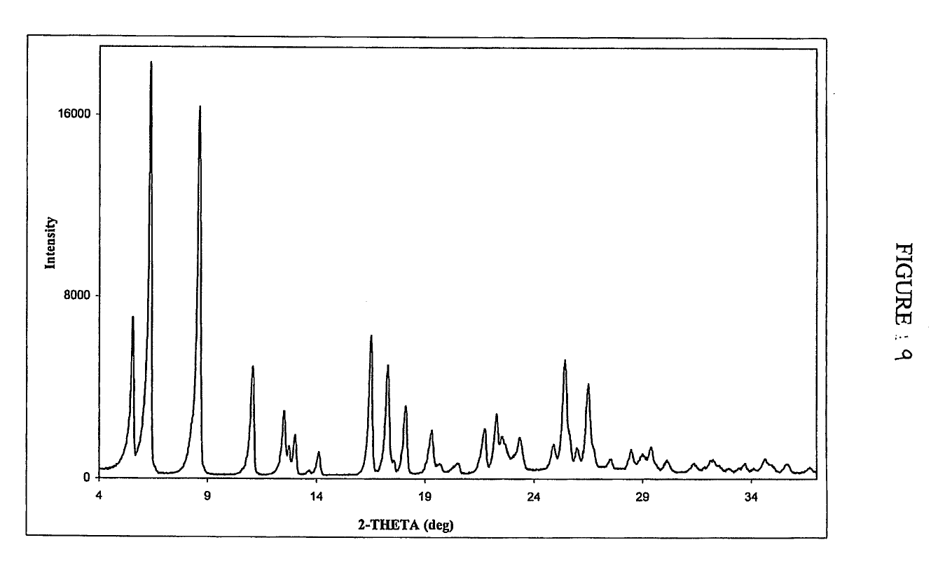

Furthermore, in one embodiment of the present invention, the crystalline form of torsemide hydrochloride is a crystalline form III of torsemide hydrochloride, characterized by a powder x-ray diffraction pattern substantially as represented in FIG. 9.

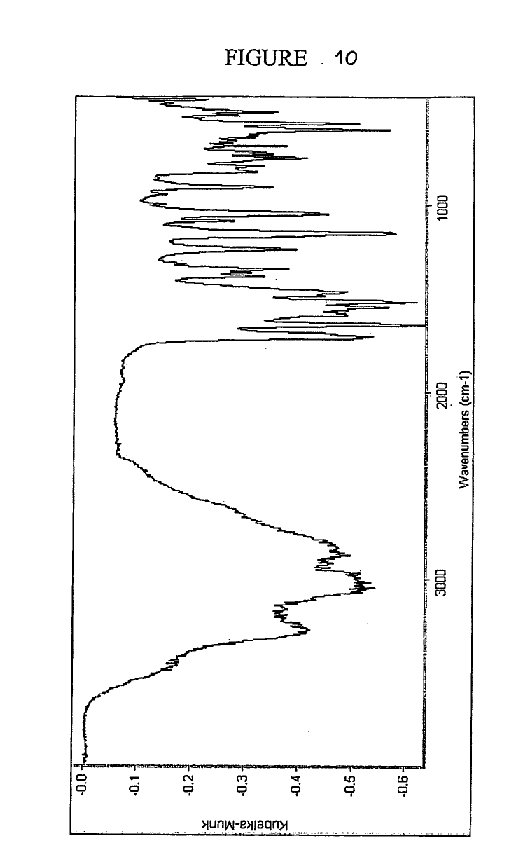

Furthermore, in one embodiment of the present invention, the crystalline form of torsemide hydrochloride is a crystalline form III of torsemide hydrochloride, characterized by an IR absorption spectrum substantially as represented in FIG. 10.

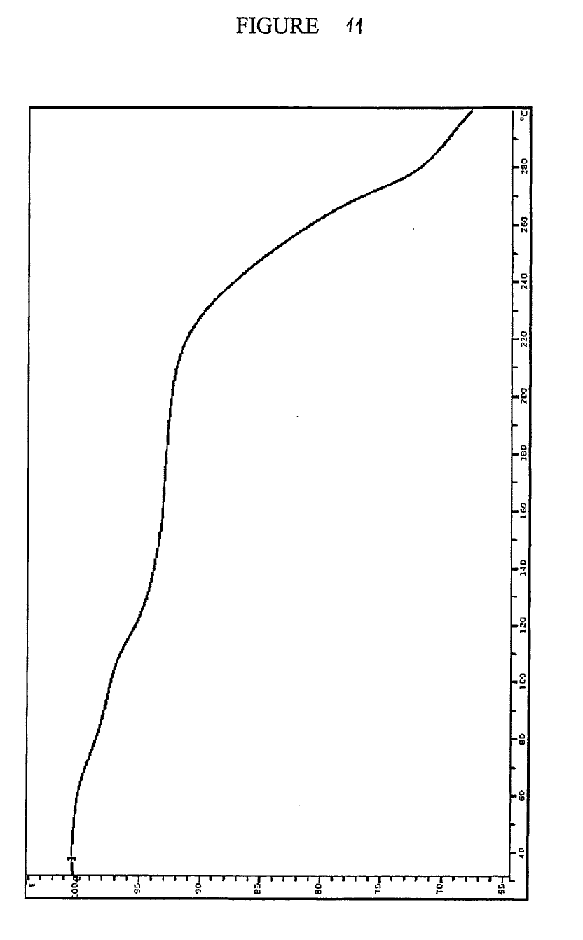

Furthermore, in one embodiment of the present invention, the crystalline form of torsemide hydrochloride is a crystalline form III of torsemide hydrochloride, characterized by the TGA as represented in FIG. 11.

In addition, in one embodiment, the present invention provides a process for the preparation of torsemide hydrochloride in a solid form comprising the step of contacting torsemide or a salt thereof with hydrogen chloride. In another embodiment, the hydrogen chloride is a gaseous hydrogen chloride. In another embodiment, the hydrogen chloride is a liquid hydrogen chloride. In another embodiment, the hydrogen chloride is a solution comprising an organic solvent, water or a combination thereof.

In addition, in one embodiment, the present invention provides a process for the preparation of the crystalline form of torsemide hydrochloride comprising the step of adding at least one solvent to a torsemide hydrochloride solution in an amount sufficient to induce the formation of crystalline torsemide hydrochloride precipitate, thereby obtaining the crystalline form of torsemide hydrochloride.

In one embodiment of the invention, the torsemide hydrochloride solution is prepared by dissolving torsemide or a salt thereof in hydrogen chloride solution which comprises an organic solvent, water or a combination thereof.

In one embodiment of the invention, the organic solvent comprises a C₁-C₃ alcohol. In another embodiment, the organic solvent comprises a C₁-C₃ acid. In another embodiment, the organic solvent comprises an ester group. In another embodiment, the organic solvent comprises an ether group. In another embodiment, the organic solvent comprises any combination of a C₁-C₃ alcohol, a C₁-C₃ acid, an ester and an ether group. In another embodiment, the organic solvent comprises methanol. In another embodiment, the organic solvent comprises acetic acid. In another embodiment, the organic solvent comprises ethyl acetate. In another embodiment, the organic solvent comprises diethyl ether. In another embodiment, the organic solvent comprises ethanol. In another embodiment, the organic solvent comprises isopropanol. In another embodiment, the organic solvent comprises any combination of methanol, acetic acid, ethyl acetate, ethanol, isopropanol or diethyl ether.

In one embodiment of the invention, the process further comprising the step of (re)crystallizing the crystalline form of torsemide hydrochloride. In another embodiment, the process further comprising the step of trituring the crystalline form of torsemide hydrochloride. In another embodiment, the process further comprising the step of reslurring the crystalline form of torsemide hydrochloride. In another embodiment, the process further comprising the step (re)crystallizing, trituring and/or reslurring the crystalline form of torsemide hydrochloride.

In one embodiment of the invention the salt is a potassium salt of torsemide. In another embodiment, the salt is a sodium salt of torsemide. In another embodiment, the salt is a lithium salt of torsemide. In another embodiment, the salt is an ammonium salt of torsemide. In another embodiment, the salt is a hydrochloride salt of torsemide. In another embodiment, the salt is any combination of potassium, sodium, lithium, ammonium or hydrochloride.

In addition, in one embodiment, the present invention provides a process for the preparation of the crystalline form II of torsemide hydrochloride comprising the step of adding a solvent mixture which comprises DMSO to a torsemide hydrochloride solution in an amount sufficient to induce the formation of crystalline torsemide hydrochloride precipitate, thereby obtaining the crystalline form II of torsemide hydrochloride.

In one embodiment of the present invention, the mixture further comprising a solvent selected from the group consisting of water, ethanol, acetone, dioxane and acitonitrile in a predefined ratio.

In addition, in one embodiment, the present invention provides a process for the preparation of the crystalline form I of torsemide hydrochloride comprising the step of adding water to a torsemide hydrochloride solution in an amount sufficient to induce the formation of crystalline torsemide hydrochloride Form I precipitate, thereby obtaining the crystalline form I of torsemide hydrochloride.

In addition, in one embodiment, the present invention provides a process for the preparation of the crystalline form III of torsemide hydrochloride comprising the step of adding at least one water miscible organic solvent to a torsemide hydrochloride solution in an amount sufficient to induce the formation of crystalline torsemide hydrochloride Form III precipitate, thereby obtaining the crystalline form III of torsemide hydrochloride.

In one embodiment of the present invention, the organic solvent comprises a ketone group. In another embodiment, the ketone is acetone.

In one embodiment of the present invention, the process for the preparation of a torsemide hydrochloride comprises the step of precipitating torsemide hydrochloride from a solution of a torsemide hydrochloride. In another embodiment, the process for the preparation of a torsemide hydrochloride comprises the step of spray drying a solution of a torsemide hydrochloride. In another embodiment, the process for the preparation of a torsemide hydrochloride comprises the step of freeze drying a solution of a torsemide hydrochloride. In another embodiment, the process for the preparation of a torsemide hydrochloride comprises the step of evaporating a solution of a torsemide hydrochloride. In another embodiment, the process for the preparation of a torsemide hydrochloride comprises the step of solidification of melts of a torsemide hydrochloride.

In one embodiment of the invention, the process for the preparation of a torsemide hydrochloride comprises the step of crystallizing or re-crystallizing the torsemide hydrochloride from a solution of a torsemide hydrochloride. In another embodiment the step of crystallizing or re-crystallizing is performed on a commercial scale in a reproducible manner.

In one embodiment of the present invention, the torsemide or the salts thereof used as starting materials in the processes for the preparation of torsemide hydrochloride as described herein may contain impurities. There is no need for a pure form of torsemide to be used as a starting material in the preparation of the torsemide hydrochloride salt.

In one embodiment of the present invention, the torsemide used as starting materials in the processes for the preparation of torsemide hydrochloride as described herein may be provided as prepared according to the procedures generally outlined in Arzneim.-Forsch./Drug Res., 1988, v. 38, 144 and U.S. Pat. No. 4,018,929 the contents of which are included herein by way of reference.

In one embodiment of the present invention, seeds may be used to initiate, encourage or facilitate crystallization. In another embodiment, the seeds are produced from a standard manufacturing run and typically have a purity in the range 96 to 99% or greater.

The torsemide hydrochloride may be obtained as a solvate, when during isolation from solution it becomes associated with the solvent in which it is dissolved. Any such solvate form is a further aspect of this invention. In a non-limiting example for the formation of a solvate of torsemide hydrochloride the crystallization of torsemide hydrochloride from acetonitrile may result in the formation of a 1:1 solvate. Solvates may be returned to the unsolvated torsemide hydrochloride by heating, for example by oven drying, or by treatment with a displacement solvent, which does not form a solvate. In one embodiment of the present invention, individual polymorphs may be crystallized directly from a solution of torsemide hydrochloride salt. In another embodiment, recrystallizing a solution of one polymorph using seeds of another polymorph may also be carried out.

The crystalline torsemide hydrochloride prepared according to the methods provided herein is sufficiently pure and may be used as a pharmaceutical compound per se or as a chemical intermediate in the preparation of other torsemide forms. In addition, according to embodiments of the present invention, a method for the optional additional purification of torsemide hydrochloride by recrystallization is provided. Such a method may also be used to provide a solid state form torsemide hydrochloride having a particular desired properties and particle size distribution.

In one embodiment, the present invention provides a method for purifying crude torsemide or a salt thereof from related impurities comprising the steps of contacting crude torsemide or a salt thereof with hydrogen chloride to obtain torsemide hydrochloride and (re)crystallizing, trituring and/or reslurring the torsemide hydrochloride to obtain a substantially pure crystalline torsemide hydrochloride.

In one embodiment, the present invention provides a method for purifying crude torsemide or a salt thereof from related impurities comprising the steps of contacting crude torsemide or a salt thereof with hydrogen chloride to obtain torsemide hydrochloride and (re)crystallizing the torsemide hydrochloride to obtain a substantially pure crystalline torsemide hydrochloride.

In one embodiment, the present invention provides a method for purifying crude torsemide or a salt thereof from related impurities comprising the steps of contacting crude torsemide or a salt thereof with hydrogen chloride to obtain torsemide hydrochloride and trituring the torsemide hydrochloride to obtain a substantially pure crystalline torsemide hydrochloride.

In one embodiment, the present invention provides a method for purifying crude torsemide or a salt thereof from related impurities comprising the steps of contacting crude torsemide or a salt thereof with hydrogen chloride to obtain torsemide hydrochloride and reslurring the torsemide hydrochloride to obtain a substantially pure crystalline torsemide hydrochloride.

In one embodiment of the present invention, substantially pure crystalline torsemide hydrochloride is 80-100% pure. In another embodiment, substantially pure crystalline torsemide hydrochloride is 80-90% pure. In another embodiment, substantially pure crystalline torsemide hydrochloride is 90-100% pure. In another embodiment, substantially pure crystalline torsemide hydrochloride is 90-97% pure.

Furthermore, in accordance to embodiments of the present invention, the compounds of this invention may be used to treat and prevent the following disorders: hypertension, oedema due to congestive heart failure and hepatic, pulmonary and renal oedema. These disorders are herein after referred to as “the Disorders”.

In on embodiment, the present invention further provides a method for treating and/or preventing any one or more of the Disorders by administering an effective and/or prophylactic amount of a torsemide hydrochloride salt of the invention to a sufferer in need thereof.

Furthermore, in one embodiment, the present invention provides a pharmaceutical composition comprising the hydrochloride salt of torsemide and a pharmaceutically acceptable carrier.

In one embodiment, the present invention provides a pharmaceutical composition comprising torsemide hydrochloride in a solid form and a pharmaceutically acceptable carrier.

In one embodiment, the present invention provides a pharmaceutical composition comprising the amorphous torsemide hydrochloride and a pharmaceutically acceptable carrier.

In one embodiment, the present invention provides a pharmaceutical composition comprising the crystalline form of torsemide hydrochloride and a pharmaceutically acceptable carrier.

In one embodiment, the present invention provides a pharmaceutical composition comprising the solvent adduct of torsemide hydrochloride and a pharmaceutically acceptable carrier.

In one embodiment, the present invention provides a pharmaceutical composition comprising the hydrate of torsemide hydrochloride and a pharmaceutically acceptable carrier.

In one embodiment, the present invention provides a pharmaceutical composition comprising the crystalline torsemide hydrochloride, characterized by a powder x-ray diffraction pattern comprising the peak at about 6.0±0.3 degrees two-theta, and a pharmaceutically acceptable carrier.

In one embodiment, the present invention provides a pharmaceutical composition comprising the crystalline form II torsemide hydrochloride, characterized by a powder x-ray diffraction pattern substantially as represented in FIG. 2, and a pharmaceutically acceptable carrier.

In one embodiment, the present invention provides a pharmaceutical composition comprising the crystalline form II torsemide hydrochloride, characterized by a single crystal x-ray crystallographic analysis with crystal parameters that are approximately equal to the following:

| Space group | P-1 |
|---|---|
| Cell dimensions | |
| a (Å) | 11.691 |
| b (Å) | 16.649 |
| c (Å) | 18.130 |
| α (°) | 62.79 |
| β (°) | 71.25 |
| γ (°) | 79.44 |
| V (Å³) | 2969.1 |
| Z (molecules/units cell) | 6 |
| Density (g/cm³) | 1.288 |

wherein a, b and c represent the lengths of the sides of the unit cell; α, β and γ represent the relative angles of the cell sides; V represents the volume of the cell; and Z represents the number of molecules in the cell unit.

In one embodiment, the present invention provides a pharmaceutical composition comprising the crystalline form I torsemide hydrochloride, characterized by a powder x-ray diffraction pattern substantially as represented in FIG. 6, and a pharmaceutically acceptable carrier.

In one embodiment, the present invention provides a pharmaceutical composition comprising the crystalline form III torsemide hydrochloride, characterized by a powder x-ray diffraction pattern substantially as represented in FIG. 9, and a pharmaceutically acceptable carrier.

In one embodiment, the present invention further provides a pharmaceutical composition for use in the treatment and/or prevention of the Disorders, which comprises an admixture of a torsemide hydrochloride salt of the invention with a pharmaceutically acceptable carrier. In another embodiment, the present invention also provides the use of a salt of the invention for treating and/or preventing the Disorders. In another embodiment, the present invention also provides the use of a salt of the invention in the manufacture of a medicament for treating and/or preventing the Disorders.

In one embodiment, the compositions of this invention may be adapted for oral administration, but formulations for dissolution for parental administration are also within the scope of this invention. In one embodiment, the composition of this invention may be presented as a unit dose composition containing between 1 and 200 mg of active ingredient calculated on a free base basis. In another embodiment, the composition may be presented as a unit dose composition containing between 2.5 and 100 mg of active ingredient calculated on a free base basis. In another embodiment, the composition may contain 2.5 mg of active ingredient calculated on a free base basis. In another embodiment, the composition may contain 5 mg of active ingredient calculated on a free base basis. In another embodiment, the composition may contain 10 mg of active ingredient calculated on a free base basis. In another embodiment, the composition may contain 20 mg of active ingredient calculated on a free base basis. In another embodiment, the composition may contain 100 mg of active ingredient calculated on a free base basis. In one embodiment, such a composition may be taken from 1 to 6 times daily for example 2, 3 or 4 times daily so that the total amount of active agent administered is within the range 2.5 to 400 mg of active ingredient calculated on a free base basis. In another embodiment, a suitable daily dose may be from 0.02 to 6 mg/kg. In another embodiment, a suitable daily dose may be from 0.07 to 0.86 mg/kg. In another embodiment, the unit dose is taken once a day.

For the desired quality-determining parameter of the form of administration according to embodiments of the present invention, the active material torsemide hydrochloride is used with the particle size distribution of at least 90%<96 μm and at least 50%<48 μm.

In one embodiment of the present invention the unit dosage forms include tablets or capsules. The compositions of this invention may be formulated by conventional methods of admixture such as blending, filling and compressing.

In one embodiment, suitable carriers for use in this invention may include a diluents, a binder, a disintegrate, a coloring agent, a flavoring agent and/or preservative. In another embodiment, the carrier for use in this invention comprises a disintegrate. Such disintegrate will be present in an effective amount, for example up to 30% by weight of the composition, to ensure disintegration of the composition in vivo. In another embodiment, the carrier for use in this invention comprises a binder. In another embodiment, the carrier for use in this invention comprises a coloring agent. Such coloring agent may be used to color a tablet coating. Commonly used coloring agents are ‘lakes’ which are largely water insoluble forms of synthetic water soluble dyes. They may be prepared by adsorbing sodium or potassium salt of a dye onto a very fine substrate of hydrated alumina, followed by treatment with a further soluble aluminum salt. The lake is then purified and dried. Examples of suitable lakes include yellow colored lakes such as sunset yellow and quinoline yellow; red colored lakes e.g. helindone pink; blue colored lakes e.g. indigotine; or mixtures thereof. Suitably compositions of the present invention comprise an amount of coloring agent sufficient to color the dosage form e.g. 0.001-1.0% w/w. Suitably the carrier for use in this invention comprises a flavoring agent. Suitably the carrier for use in this invention comprises a preservative.

In a further embodiment, the present invention provides a pack comprising a pharmaceutical composition according to the present invention.

This invention is further illustrated in the Experimental Details section, which follows. This section is set forth to aid in an understanding of the invention but is not intended to, and should not be construed to; limit in any way the invention as set forth in the claims that follow thereafter.

### EXPERIMENTAL DETAILS

HPLC was carried out on a Merck-Hitachi Lachrom chromatographic system with UV detector.

Single crystal x-ray crystallographic analysis was performed on a Phillips PW 11000 diffractometer, ω/2θ mode, graphite monochromator, MoK_{α} radiation.

Powder x-ray diffraction patterns were obtained by methods known in the art using a Philips analytical x-ray powder diffractometer for wide-angle x-ray diffraction (CuK_{α} radiation of γ=1.5418 Å, monochromator before detector, Pw3020 goniometer system). The Bragg-Brentano scheme was used for beam focusing.

¹H spectra were recorded on a Bruker AM-200 (200 MHz) and Bruker AM400 (400 MHz) instruments using DMSO as a solvent.

Melting points were determined in open capillary tubes with Büchi B-545. The melting points of torsemide hydrochloride generally depend upon their crystalline form and level of purity.

Infrared spectra were recorded on a Nicolet Impact 410 FT-IR spectrophotometer equipped with Diffuse Reflectance accessory using a 5% dispersion of sample material in a potassium bromide over the wave number range 4000 to 400 cm⁻¹. A Kubelka-Munk conversion was used.

DSC curves were recorded on a Mettler-Toledo DSC 822e Differential Scanning Calorimeter.

TGA curves were recorded on a Mettler-Toledo TGA/SDTA851e Thermogravimetric Analyzer.

### Example 1

### Preparation of Torsemide Hydrochloride Form II

A mixture of DMSO (2 mL) and water (10 mL) was added dropwise to a stirred solution of torsemide with 98% purity by HPLC (1.5 g) in 37% hydrochloric acid (4 mL). The mixture was kept overnight at 20-25° C. The precipitated solid was filtered off and dried under reduced pressure to give torsemide hydrochloride as white crystals of Form II with mp 165-167° C. and 99.5% purity by HPLC.

The crystals of torsemide hydrochloride Form II were characterized by powder x-ray and IR absorption analysis as set forth above and in FIGS. 2 and 4.

Single crystal of the torsemide hydrochloride Form II was isolated and used for determination crystallographic parameters (Tables 1-6).

### Example 2

### Preparation of Torsemide Hydrochloride Form II

A mixture of DMSO (1.3 mL) and absolute ethanol (3 mL) was added dropwise to a stirred solution of torsemide (0.6 g) in 37% hydrochloric acid (2 mL). The mixture was stirred for 2 hours at room temperature and kept overnight without stirring at the same temperature. The precipitated solid was filtered off, washed on the filter with ice-cold absolute ethanol (2×3 mL) and dried under reduced pressure to a constant weight to give 0.5 g of torsemide hydrochloride Form II.

### Example 3

### Preparation of Torsemide Hydrochloride Form II

A mixture of DMSO (1.5 mL) and acetone (1.5 mL) was added dropwise to a stirred solution of torsemide (0.6 g) in 37% hydrochloric acid (2 mL). The resulting mixture was stirred for 2 hours at room temperature. The precipitated solid was filtered off and dried under reduced pressure to give torsemide hydrochloride Form II.

### Example 4

### Preparation of Torsemide Hydrochloride Form II

A mixture of DMSO (1 mL) and dioxane (1 mL) was added to a solution of torsemide (0.6 g) in 37% hydrochloric acid (2 mL) and white solid was formed. The resulting suspension was stirred for 1 hour at room temperature. The precipitated solid was filtered off, washed on the filter with ice-cold dioxane and dried under reduced pressure to give 0.5 g of torsemide hydrochloride Form II.

### Example 5

### Preparation of Torsemide Hydrochloride Form II

A mixture of DMSO (1 mL) and acetonitrile (1 mL) was added dropwise to a solution of torsemide (0.6 g) in 37% hydrochloric acid (2 mL) and the resulting mixture was kept overnight at room temperature. The precipitated solid was filtered off and dried under reduced pressure to give torsemide hydrochloride Form II.

### Example 6

### Preparation of Torsemide Hydrochloride Form II

A mixture of DMSO (1 mL) and water (6 mL) was added dropwise to a stirred solution of lithium torsemide (0.5 g) in 37% hydrochloric acid (2 mL) and the resulting mixture was stirred for 1 hour at room temperature. The precipitated solid was filtered off and dried under reduced pressure to give torsemide hydrochloride Form II.

### Example 7

### Preparation of Torsemide Hydrochloride Form I

A mixture of hexane (5 mL) and water (6 mL) was added to a stirred solution of torsemide (0.6 g) in 37% hydrochloric acid (2 mL). The resulting mixture was stirred for 1 hour at room temperature. The precipitated solid was filtered off and dried under reduced pressure to give 0.35 g of torsemide hydrochloride Form I.

### Example 8

### Preparation of Torsemide Hydrochloride Form I

A mixture of isopropanol (1 mL) and water (1.3 mL) was added to a solution of torsemide (0.6 g) in 37% hydrochloric acid (2 mL) and the resulting mixture was stirred overnight at room temperature. The precipitated solid was filtered off, washed on the filter with ice-cold isopropanol and dried under reduced pressure to give 0.17 g of torsemide hydrochloride Form I.

### Example 9

### Preparation of Torsemide Hydrochloride Form III

Acetone (10 mL) was added dropwise to a solution of torsemide (0.6 g) in 37% hydrochloric acid (2 mL). The resulting mixture was stirred overnight at room temperature and acetone was removed in vacuum until white precipitate appeared in flash. The precipitated solid was filtered off, and dried under reduced pressure to give torsemide hydrochloride Form III.

It will be appreciated that the present invention is not limited by what has been described hereinabove and that numerous modifications, all of which fall within the scope of the present invention, exist. Rather the scope of the invention is defined by the claims that follow:

1. A hydrochloride salt of torsemide in a solid form.

2. Torsemide of claim 1, wherein solid form is an amorphous torsemide hydrochloride.

3. Torsemide of claim 1, wherein solid form is a crystalline form of torsemide hydrochloride.

4. Torsemide of claim 1, wherein solid form is a solvent adduct of torsemide hydrochloride.

5. Torsemide of claim 1, wherein solid form is a hydrate of torsemide hydrochloride.

6. The crystalline form of torsemide hydrochloride according to claim 3, characterized by a powder x-ray diffraction pattern comprising the peak at about 6.0±0.3 degrees two-theta.

7. The crystalline form of torsemide hydrochloride according to claim 3, wherein said crystalline form is a crystalline form II of torsemide hydrochloride, characterized by a powder x-ray diffraction pattern substantially as represented in FIG. 2.

8. The crystalline form of torsemide hydrochloride according to claim 3, wherein said crystalline form is a crystalline form II of torsemide hydrochloride, characterized by a single crystal x-ray crystallographic analysis with crystal parameters that are approximately equal to the following:

| Space group | P-1 |
|---|---|
| Cell dimensions | |
| a (Å) | 11.691 |
| b (Å) | 16.649 |
| c (Å) | 18.130 |
| α (°) | 62.79 |
| β (°) | 71.25 |
| γ (°) | 79.44 |
| V (Å³) | 2969.1 |
| Z (molecules/units cell) | 6 |
| Density (g/cm³) | 1.288 |

wherein a, b and c represent the lengths of the sides of the unit cell; α, β and γ represent the relative angles of the cell sides; V represents the volume of the cell; and Z represents the number of molecules in the cell unit.

9. The crystalline form of torsemide hydrochloride according to claim 3, wherein said crystalline form is a crystalline form I of torsemide hydrochloride, characterized by a powder x-ray diffraction pattern substantially as represented in FIG. 6.

10. The crystalline form of torsemide hydrochloride according to claim 3, wherein said crystalline form is a crystalline form III of torsemide hydrochloride, characterized by a powder x-ray diffraction pattern substantially as represented in FIG. 9.

11. A process for the preparation of torsemide of claim 1 comprising the step of contacting torsemide or a salt thereof with hydrogen chloride.

12. The process according to claim 11, wherein said hydrogen chloride is a gaseous hydrogen chloride.

13. The process according to claim 11, wherein said hydrogen chloride is a hydrogen chloride solution, wherein said solution comprises an organic solvent, water or a combination thereof.

14. The process according to claim 11, wherein said salt is a potassium, sodium, lithium, ammonium salt of torsemide or any combination thereof.

15. A process for the preparation of the crystalline form of torsemide hydrochloride of claim 3 comprising the step of:
adding at least one solvent to a torsemide hydrochloride solution in an amount sufficient to induce the formation of crystalline torsemide hydrochloride precipitate;
thereby obtaining said crystalline form of torsemide hydrochloride.

16. The process according to claim 15, wherein said torsemide hydrochloride solution is prepared by dissolving torsemide or a salt thereof in hydrogen chloride solution which comprises an organic solvent, water or a combination thereof.

17. The process according to claim 16, wherein said organic solvent comprises a C₁-C₃ alcohol, a C₁-C₃ acid, an ester, an ether group or any combination thereof.

18. The process according to claim 17, wherein said organic solvent is methanol, acetic acid, ethyl acetate, diethyl ether or any combination thereof.

19. The process according to claim 15, further comprising the step of (re)crystallizing, trituring and/or reslurring said crystalline form of torsemide hydrochloride.

20. The process according to claim 16, wherein said salt is a potassium, sodium, lithium, ammonium, hydrochloride salt of torsemide or any combination thereof.

21. A process for the preparation of the crystalline form II of torsemide hydrochloride comprising the step of:
adding a solvent mixture which comprises DMSO to a torsemide hydrochloride solution in an amount sufficient to induce the formation of crystalline torsemide hydrochloride precipitate;
thereby obtaining said crystalline form II of torsemide hydrochloride.

22. The process according to claim 21, wherein said mixture further comprising a solvent selected from the group consisting of water, ethanol, acetone, dioxane and acitonitrile in a predefined ratio.

23. A process for the preparation of the crystalline form I of torsemide hydrochloride, according to claim 9 comprising the step of:
adding water to a torsemide hydrochloride solution in an amount sufficient to induce the formation of crystalline torsemide hydrochloride Form I precipitate;
thereby obtaining said crystalline form I of torsemide hydrochloride.

24. A process for the preparation of the crystalline form III of torsemide hydrochloride, according to claim 10 comprising the step of:
adding at least one water miscible organic solvent to a torsemide hydrochloride solution in an amount sufficient to induce the formation of crystalline torsemide hydrochloride Form III precipitate;
thereby obtaining said crystalline form III of torsemide hydrochloride.

25. The process according to claim 24, wherein said organic solvent comprises a ketone group.

26. The process according to claim 25, wherein said ketone is acetone.

27. A method for purifying crude torsemide or a salt thereof from related impurities comprising the steps of:
contacting crude torsemide or a salt thereof with hydrogen chloride to obtain torsemide hydrochloride; and
(re)crystallizing, trituring and/or reslurring said torsemide hydrochloride to obtain a substantially pure crystalline torsemide hydrochloride.

28. A pharmaceutical composition comprising the hydrochloride salt of torsemide according to claim 1 and a pharmaceutically acceptable carrier.

29. A pharmaceutical composition comprising the amorphous torsemide hydrochloride according to claim 2 and a pharmaceutically acceptable carrier.

30. A pharmaceutical composition comprising the crystalline form of torsemide hydrochloride according to claim 3 and a pharmaceutically acceptable carrier.

31. A pharmaceutical composition comprising the solvent adduct of torsemide hydrochloride according to claim 4 and a pharmaceutically acceptable carrier.

32. A pharmaceutical composition comprising the hydrate of torsemide hydrochloride according to claim 5 and a pharmaceutically acceptable carrier.

33. A pharmaceutical composition comprising the crystalline torsemide hydrochloride according to claim 6 and a pharmaceutically acceptable carrier.

34. A pharmaceutical composition comprising the crystalline form II of torsemide hydrochloride according to claim 7 and a pharmaceutically acceptable carrier.

35. A pharmaceutical composition comprising the crystalline form II of torsemide hydrochloride according to claim 8 and a pharmaceutically acceptable carrier.

36. A pharmaceutical composition comprising the crystalline form I of torsemide hydrochloride according to claim 9 and a pharmaceutically acceptable carrier.

37. A pharmaceutical composition comprising crystalline form III of torsemide hydrochloride according to claim 10 and a pharmaceutically acceptable carrier.
